(19) United States
(12) Patent Application Publication
(10) Pub. No.: US 2019/0150861 A1
(43) Pub. Date: May 23, 2019

(54) Gold is a Complementary Contrast Agent for Spectral CT

(71) Applicant: BOARD OF REGENTS, THE UNIVERSITY OF TEXAS SYSTEM, Austin, TX (US)

(72) Inventors: Matthew A. LEWIS, Farmers Branch, TX (US); Khaled A. NASR, Dallas, TX (US); Todd C. SOESBE, Dallas, TX (US)

(21) Appl. No.: 16/097,414

(22) PCT Filed: Apr. 28, 2017
(86) PCT No.: PCT/US17/30048

§ 371 Date: Oct. 29, 2018

Related U.S. Application Data
(60) Provisional application No. 62/329,575, filed on Apr. 29, 2016.

(51) Int. Cl. A61B 6/03; A61K 49/04; A61B 6/00

(52) U.S. Cl. CPC A61B 6/032; A61B 6/481; A61K 49/04

(57) ABSTRACT

The present invention includes a method of resolving gold from iodine using a multi-detector Spectral Computed Tomography (CT) scanner comprising: obtaining an image using the scanner of a subject provided a gold contrast agent; performing a Spectral CT material differentiation or imaging at 140 kVp using one or more scanner tubes; and calculating a DE ratio for ratio of gold, wherein the DE ratio is about 1.0.

### TECHNICAL FIELD OF THE INVENTION

The present invention relates in general to the field of contrast agents, and more particularly, to using gold as a nearly perfect complementary contrast agents at 140 kVp in a multi-detector Spectral CT, e.g., the Philips IQon Spectral CT.

### BACKGROUND OF THE INVENTION

Without limiting the scope of the invention, its background is described in connection with gold as a contract agent.

Krissak, et al., in an article entitled “Gold as a Potential Contrast Agent for Dual-Energy CT”, Advances in Molecular Imaging, 2013, 3, 37-42, review images of gold on a Siemens dual source CT scanners with a variety of image parameters and surprisingly report the DE ratio of gold is independent of x-ray energies. Specifically, Krissak reports that the DE ratio for gold to be less than 1.0 measured during gold-iodine decomposition. This article describes dual energy CT and gold as a contrast agent at 80, 100, 120, and 140 kVp using a single scanner tube. The article reports the DE ratio of gold as 0.92 at 140 kVp.

Nowak, et al., in an article entitled “Potential of high-Z contrast agents in clinical contrast-enhanced computed tomography”, Medical Physics (2011), 38(12), 6469-6482, discuss iodine- and barium-based contrast media (CM) are used as a clinical contrast-enhanced computed tomography (CE-CT). The purpose of their study was to quantify the potential for dose reduction when using CM based on heavy metals, e.g., iodine, holmium, gadolinium, ytterbium, osmium, tungsten, gold, and bismuth. It is said that even higher-Z materials such as gold and bismuth showed a good overall performance in conjunction with high tube voltage, large patients or strong added filtration and may be recommended for scans under these conditions.

### SUMMARY OF THE INVENTION

In one embodiment, the present invention includes a method of resolving gold from iodine using a multi-detector Spectral Computed Tomography (CT) scanner comprising: obtaining an image using the scanner of a subject provided a gold contrast agent; performing a Spectral CT material differentiation or imaging at 140 kVp using one or more scanner tubes; and calculating a dual energy (DE) ratio for ratio of gold, wherein the DE ratio is about 1.0. In one aspect, the step of performing a Spectral CT material differentiation or imaging at 140 kVp is conducted using two, three, or four scanner tubes. In another aspect, the method further comprises the step of providing a subject with 0.1, 0.5, 1.0, 1.0, 2.5 or 5.0 mg/ml of gold contrast agent. In another aspect, the gold is detected as a super-dense water in water-iodine decomposition. In another aspect, the differentiation of the gold-iodine measured is not spectral diffusion. In another aspect, the gold is imaged at 140 kVp/255 mAs. In another aspect, the contrast due to gold is independently from contrast due to iodine in the common Iodine/Water material decomposition. In another aspect, the method further comprises the step of generating an image of the gold.

In another embodiment, the present invention includes a method of resolving gold from iodine using a Philips IQon Spectral Computed Tomography (CT) scanner comprising: obtaining an image using the scanner of a subject provided a gold contrast agent; performing a Spectral CT material differentiation or imaging at 140 kVp using two or more scanner tubes; and calculating a dual energy (DE) ratio for ratio of gold, wherein the DE ratio is about 1.0, wherein the contrast due to gold is independently from contrast due to iodine in the common Iodine/Water material decomposition. In one aspect, the step of performing a Spectral CT material differentiation or imaging at 140 kVp is conducted using three or four scanner tubes. In another aspect, the method further comprises the step of providing a subject with 0.1, 0.5, 1.0, 1.0, 2.5 or 5.0 mg/ml of gold contrast agent. In another aspect, the gold is detected as a super-dense water in water-iodine decomposition. In another aspect, the differentiation of the gold-iodine measured is not spectral diffusion. In another aspect, the gold is imaged at 140 kVp/255 mAs.

### BRIEF DESCRIPTION OF THE DRAWINGS

For a more complete understanding of the features and advantages of the present invention, reference is now made to the detailed description of the invention along with the accompanying figures and in which:

FIG. 1 is a graph that shows the location of AuCl₂ in various concentrations in the Photoelectric/Compton material decomposition space (open squares). Open circles indicate locations for 5 mg/ml solutions of elements near gold.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

Spectral CT imaging, in its various forms (dual energy CT—DECT, dual source CT—DSCT, spectral detector CT—SDCT), is inherently more stable in terms of CT number. When the sinograms are close to ideally registered (as is the case with SDCT and rapid kV switching DECT), then a projection space-based decomposition using the Alvarez-Macovski Photoelectric/Compton basis pair is optimal. For tissues that are well-modeled by a linear combination of the PE and Compton energy dependence, the dual energy reconstructions will be ideally independent of changes in the x-ray tube energy (kVp). That said, high-Z elements with significant K-edges in the diagnostic energy range will produce interesting energy-dependent artifacts in the spectral reconstruction process. The present inventors show herein that, surprisingly, contrast due to gold (Au, Z=79) on the Philips IQon Spectral CT at 140 kVp is near perfectly complementary to water and does not impact the corresponding quantitative iodine map.

The present inventors have determined that contrast due to gold would have special properties specifically on the Philips IQon at 140 kVp x-ray tube energy. Surprisingly, it was found that gold appears as super-dense water with negligible contrast appearing as iodine. This was experimentally confirmed using a phantom containing AuCl₂ at 140 kVp.

It was further found that, based on the properties of the Philips IQon x-ray tube and dual-layer detectors, gold is surprisingly positioned in the parameter space to behave radiographically as water. Even though it is highly attenuating on a conventional CT, it will not affect the quantitative estimation of iodine in spectral CT.

This is because gold lies exactly on the material decomposition line for water-like materials (that is, materials with same mass attenuation as water but differing only in density). This corresponds to a Dual Energy ratio (DE ratio) of 1.0. This line is at the boundary between complementarity and cloaking. At 120 kVp on the Philips IQon, gold is a cloaking high-Z contrast agent.

As such, gold can be paired with an iodinated contrast agent, a gold-based contrast agent would allow quantitative dual contrast media studies to be performed simultaneously. High-Z contrast agents described as complementary do not allow completely quantitative separation of dual contrast agents. They possess a DE ratio in the range 1.0<DE_Complementary< >DE_Iodine. In this specific case, completely quantitative dual contrast media studies are feasible since the DE ratio for gold was found to be DE=1.0.

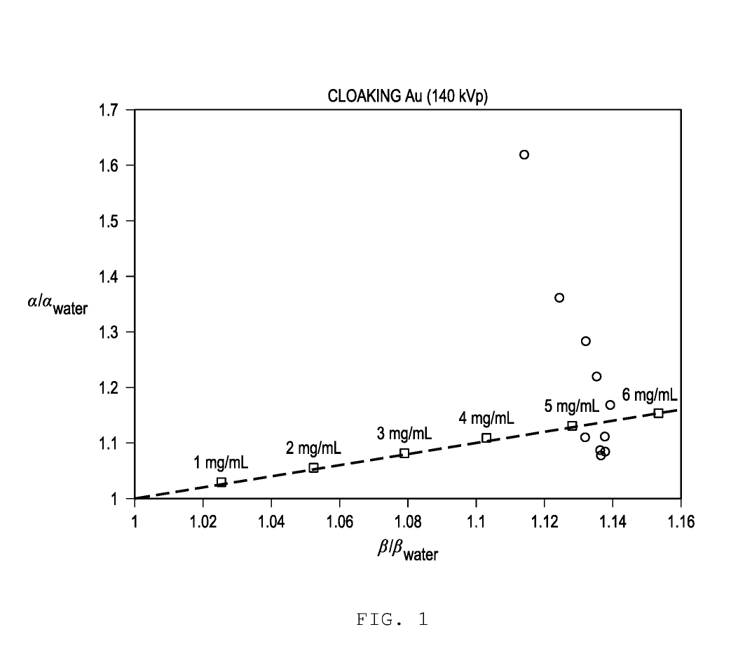

The contrast behavior of high-Z elements in a variety of spectral CT systems (DSCT, SDCT) was explored in 2D using the toySCT simulation tool. Using the results from these simulations, the model-error artifacts produced by these elements can be explained. Furthermore, from simulation as shown in FIG. 1, it was noted that at 140 kVp on the Philips IQon Spectral CT with dual-layer detectors contrast due to gold was situated on the parameter line for water-like materials. FIG. 1 is a graph that shows the location of AuCl₂ in various concentrations in the Photoelectric/Compton material decomposition space (open squares), open circles indicate locations for 5 mg/ml solutions of elements near gold. Surprisingly, it was found that gold lies on the unit line at 140 kVp on the Philips IQon Spectral CT.

The dual energy ratio (DE ratio) was predicted to be exactly 1 in this case. A phantom containing AuCl₂ in concentrations of 1.25, 2.5, and 5.0 mg/ml was imaged at 140 kVp/255 mAs. Both Water-no-Iodine (Virtual Non-Contrast in Hounsfield units) and Iodine-no-Water (mg/ml) were evaluated in the Philips Intelliscape Portal DICOM viewer. The iodine quantitative map reported iodine concentrations of 0.1±0.10, 0.0±0.05, 0.1±0.10 mg/ml. Another indication of perfect complementarity is agreement in CT Number between conventional and VNC images. CT Number was invariant between conventional and VNC in the 1.25 and 2.5 mg/ml Au vials with a slight statistical deviation at 5 mg/ml. At 120 kVp, CT Number differed between conventional and VNC for all vials, indicating a reduced level of complementarity.

The contrast behavior of high-Z elements in a variety of spectral CT systems (DSCT, SDCT) was explored in 2D using the toySCT simulation tool described elsewhere. Using the results from these simulations, the model-error artifacts produced by these elements can be explained. Furthermore, as shown in FIG. 1, it was noted that at 140 kVp on the Philips IQon Spectral CT with dual-layer detectors contrast due to gold was situated on the parameter line for water-like materials. The dual energy ratio (DE ratio) was predicted to be exactly 1 in this case. A phantom containing AuCl₂ in concentrations of 1.25, 2.5, and 5.0 mg/ml was imaged at 140 kVp/255 mAs. Both Water-no-Iodine (Virtual Non-Contrast (VNC) in Hounsfield units) and Iodine-no-Water (mg/ml) were evaluated in the Philips Intelliscape Portal DICOM viewer. The iodine quantitative map reported iodine concentrations of 0.1±0.10, 0.0±0.05, 0.1±0.10 mg/ml. Another indication of perfect complementarity is agreement in CT Number between conventional and VNC images. CT Number was invariant between conventional and VNC in the 1.25 and 2.5 mg/ml Au vials with a slight statistical deviation at 5 mg/ml. At 120 kVp, CT Number differed between conventional and VNC for all vials, indicating a reduced level of complementarity.

Gold nanoparticle-based contrast has been previously described in the literature as the foundation for potential targeted CT contrast agents. Micelle-based gold contrast has also been described. In this work, the inventors demonstrate that gold-based contrast agents possess an added benefit when imaged at 140 kVp on the Philips IQon Spectral CT with dual-layer detectors. Contrast due to gold will act independently from contrast due to iodine in the common Iodine/Water material decomposition. At present, it does not appear that this property exists for the scan parameter space available in any clinical DSCT systems. Thus, using gold as a spectral CT contrast agent, it is feasible under specific circumstances to perform dual quantitative contrast studies.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), propertie(s), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

1. A method of resolving gold from iodine using a multi-detector Spectral Computed Tomography (CT) scanner comprising:
obtaining an image using the scanner of a subject provided a gold contrast agent;
performing a Spectral CT material differentiation or imaging at 140 kVp using one or more scanner tubes; and
calculating a dual energy (DE) ratio for ratio of gold, wherein the DE ratio is about 1.0.

2. The method of claim 1, wherein the step of performing a Spectral CT material differentiation or imaging at 140 kVp is conducted using two, three, or four scanner tubes.

3. The method of claim 1, further comprising the step of providing a subject with 0.1, 0.5, 1.0, 1.0, 2.5 or 5.0 mg/ml of gold contrast agent.

4. The method of claim 1, wherein the gold is detected as a super-dense water in water-iodine decomposition.

5. The method of claim 1, wherein the differentiation of the gold-iodine measured is not spectral diffusion.

6. The method of claim 1, wherein the gold is imaged at 140 kVp/255 mAs.

7. The method of claim 1, wherein the contrast due to gold is independently from contrast due to iodine in the common Iodine/Water material decomposition.

8. The method of claim 1, further comprising the step of generating an image of the gold.

9. A method of resolving gold from iodine using a Philips IQon Spectral Computed Tomography (CT) scanner comprising:
obtaining an image using the scanner of a subject provided a gold contrast agent;
performing a Spectral CT material differentiation or imaging at 140 kVp using two or more scanner tubes; and
calculating a dual energy (DE) ratio for ratio of gold, wherein the DE ratio is about 1.0, wherein the contrast due to gold is independently from contrast due to iodine in the common Iodine/Water material decomposition.

10. The method of claim 9, wherein the step of performing a Spectral CT material differentiation or imaging at 140 kVp is conducted using three or four scanner tubes.

11. The method of claim 9, further comprising the step of providing the subject with 0.1, 0.5, 1.0, 1.0, 2.5 or 5.0 mg/ml of gold contrast agent.

12. The method of claim 9, wherein the gold is detected as a super-dense water in water-iodine decomposition.

13. The method of claim 9, wherein the differentiation of the gold-iodine measured is not spectral diffusion.

14. The method of claim 9, wherein the gold is imaged at 140 kVp/255 mAs.
